(19) United States
(12) Patent Application Publication
(10) Pub. No.: US 2003/0086818 A1
(43) Pub. Date: May 8, 2003

(54) Portable mail sterilizer which counters biological and chemical warfare agents

(76) Inventors: Merrel Truly Holley JR., Morgan City, LA (US); Donald Spencer Turnage JR., Morgan City, LA (US)

Correspondence Address: MERREL TRULY HOLLEY, JR., 1315 HWY 182, MORGAN CITY, LA, 70380, US

(21) Appl. No.: 10/013,100

(22) Filed: Nov. 6, 2001

(51) Int. Cl. A61L 2/10

(52) U.S. Cl. 422/24; 422/22; 422/186.07; 422/186.3; 422/187

(57) ABSTRACT

A process for sterilizing mail and/or packages of biological warfare agents, which will also mineralize chemical warfare agents. A machine for sterilizing mail and/or packages of biological warfare agents, which will also mineralize chemical warfare agents, having a horizontally arranged cylindrical housing with openings provided in the sidewall through which a user can insert his hands and can view the interior of the housing, a door at one end which covers a portion of one end of the housing, a UVC light source, and a photocatalytic coating. An electro-mechanical door interlock prevents the door from opening during the sterilization cycle.

### BACKGROUND

It is known to purify air and water using UVC and ozone. Such products are readily available on the market under various manufacturers labels, such as: Phillips; GE. It is also known to initiate photcatalysis within a SiO2 film.

The principal object of the present invention is to provide an improved process for sterilizing mail and/or packages to ensure life-safety in the current state of affairs in which terrorists are sending anthrax spores and other agents of biological warfare through the mail. This invention embodies a comprehensive and combinatorial approach which has a wholistic virtue vastly different and far greater than any method in common use. There is a tremendous market demand for such a technology. This process is not readily apparent. The known methods of mail sterilization are steam and dry heat autoclaving and electron beams. Electron beam systems are very expensive and are not portable. Autoclaves take longer to sterilize, damage the letters, and will not destruct chemical warfare agents which could easily be inserted into letters and/or packages. These known methods are not comprehensive, nor do they provide the same function as the present invention. The present invention creates a system that is superior to such known methods and is radically different in scope and function.

### SUMMARY

The present invention is a process for sterilizing mail and/or packages of the agents of biological and chemical warfare agents. The present invention is also the device for use in sterilizing the mail and packages of the agents of biological and chemical warfare agents. These biological agents can be bacteria or bacterial spores, such as anthrax; viral, such as smallpox; or fungal spores. Chemical agents include sarin and other nerve gases. The mail sterilizing cabinet comprises a substantially cylindrical housing oriented horizontally and having opposing (circular) end regions. One of the ends is covered completely with an endplate, and the other end is partially covered with an endplate affixed to the housing. The remainder of the area not covered with an endplate is covered with a door. The housing is preferably supported above the ground by mounting it on a frame.

Openings are also provided in the cylindrical housing, with gloves attached, into which a user can insert their hands to manipulate (open and inspect) mail or packages inside. A transparent window, typically curved so as to conform to the shape of the cylinder, is also provided in the sidewall, which permits the user to view the mail, packages, and their contents as it is opened. This window is made of a material, such as a doped polycarbonate or doped acrylic, which blocks the transmission of 100% of light at the specific wavelengths of 185 nm and 254 nm, thus protecting the eyes of the user. Seals around the endplate, electrical, door, window, and glove openings make the cabinet airtight.

The endplate of the cabinet containing the door is preferably removable therefrom in order to service interior parts. The endplate is gasketed underneath to create an airtight seal when bolted firmly in position. The door is provided with a retaining means, which engages with the inside of the partial endplate. This door is gasketed in order to be airtight when closed. A grate can be provided interior the cylindrical housing, and rests on supports, which is used to support mail and/or packages therein.

Light sources, emitting light at the UVC wavelength of 254 nm, are positioned so that the bulbs are aligned longitudinally in the cabinet. This wavelength light serves to disrupt the genetic material of all biological agents in the cabinet, thus sterilizing the chamber contents. The light sources can also be configured to emit light at 185 nm wavelength which generates ozone, a strong disinfectant. A normally open switch on the door housing, said switch opening when the door opens, de-energizing the UVC light circuit, and which switch closes when the door closes, thus energizing the UVC light circuit, ensures the safety of the user. When the door is closed, the door locking mechanism circuit becomes energized, disallowing the door from being opened until the complete sterilization cycle is completed by means of a timer.

The cylindrical shape of the cabinet housing affords several advantages. Significantly, its curved shape serves to reflect the photons not absorbed by the SiO2 coating back toward the mail and/or packages when they are positioned along the central longitudinal axis of the housing, plus all microbes are held in close proximity to the UVC with no corners for them to collect. A low velocity fan positioned so that the direction of flow is along the curved outer wall of the chamber, creates an air current that ensures sufficient contact time of all target molecules and microbes for complete sterilization. This serves to increase the efficacy. The interior surfaces of the cylindrical cabinet housing are coated in a thin film of titanium dioxide, a semi-conductor that, when excited with light at the 254 nm and 185 nm wavelengths, creates strong photocatalytic oxidation which mineralizes organic compounds, such as chemical warfare agents like sarin. The curved shape of the housing also permits the positioning of a window substantially above the mail and/or packages, which serves to improve the viewing angle for the user since they can view the object from above rather than from the side where the view may become obscured. Further aiding in the improvement of visibility is a visible spectrum light source.

In typical useage a person inserts mail into the cabinet, closes the door, inserts their arms into the gloves, and opens and inspects their mail and/or packages. The contents of the chamber are sterilized and/or mineralized are then safe to handle.

### BRIEF DESCRIPTION

Technical Field

This invention relates to the sterilization of mail and/or packages of biological agents such as anthrax, smallpox, fungal spores, etc. that may contaminate mail in bioterrorist attacks. This invention also will mineralize the organic compounds used as advanced chemical warfare agents, such as sarin. This invention is comprehensive in technologies in that microbes are sterilized by a combination of UVC and ozone, and the same wavelengths of light used for sterilization, specifically in and about 254 nm, serves to excite the SiO2 thinfilm semi-conductor coating the interior of the cylindrical shell of the sterilization chamber, which mineralizes organic molecules by photocatalysis.

### BRIEF DESCRIPTION OF THE DRAWING

FIG. 1 depicts a perspective (frontal) view of a preferred mail sterilization cabinet according to the present invention.

FIG. 2 depicts a cut-away view of the interior of a typical sterilization chamber design of the present invention.

FIG. 3 depicts a perspective view of one end of a cabinet according to the present invention in which the door is engaged with a removable endplate.

### DETAILED DESCRIPTION OF THE INVENTION

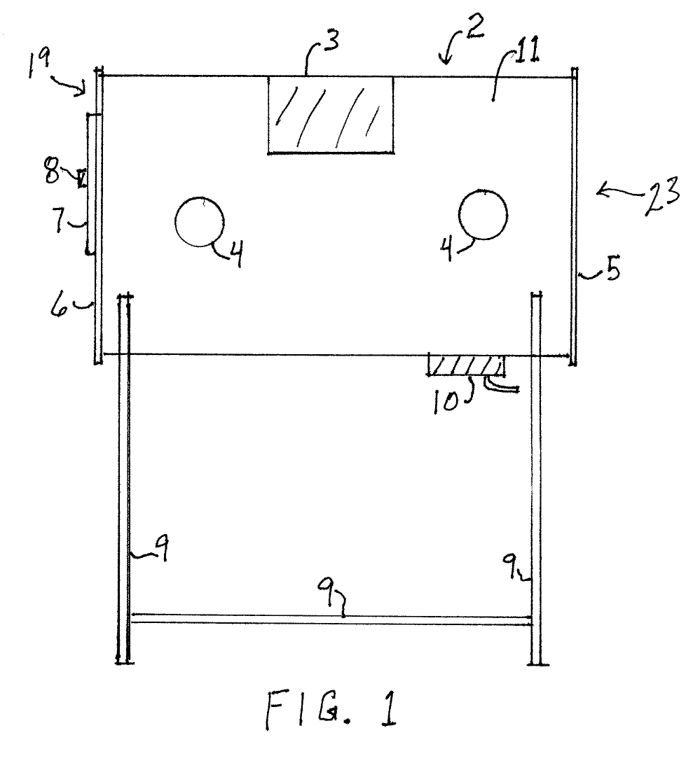

The invention will now be described in more detail with reference to the figures. Referring to FIG. 1, cabinet 2 is constructed of a cylindrical housing 11, which is mounted on a support frame 9. Cylindrical housing 11 is conveniently provided by a section of PVC pipe or air conditioning duct. Preferred dimensions for the cylindrical region of the cabinet are about 48 inches in length and 24 inches in diameter.

Cylindrical housing 11 is further provided with armholes 4 in the side of the cylindrical housing through which a user's hands can be inserted. Also an opening is provided in the side of the housing so that a user can view the interior of the housing and the mail and/or packages therein. These holes are conveniently provided in the housing with a holesaw. The view opening is provided with an observation window 3, which is preferably made of a transparent plastic sheet. A particularly preferred plastic material is doped acrylic, which has the durability and flexibility needed to conform to the housing. This plastic sheet blocks the transmission of 100% of UV light, thus protecting the eyes of the user. This type of plastic sheet can be obtained from Lase-R Shield, Inc. The plastic sheet is preferably attached to housing 1 with bolts with nuts retaining said bolts on the inside of the cylindrical housing 11. Observation window 3 is gasketed beneath to be airtight at 1 atmosphere pressure. Electrical connections are housed in electrical box 10 and unit is powered by 120 vac household current supplied by typical pronged cord. All penetrations in cylindrical housing to facilitate electrical wiring are sealed to airtightness. Also housed in electrical box 10 are the timer circuit and light ballasts.

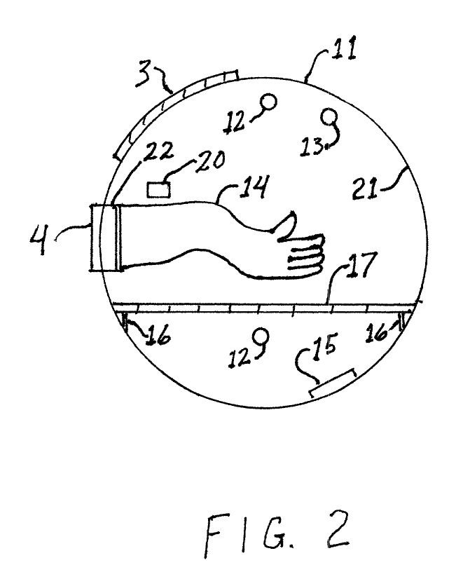

An internal view of mail sterilization cabinet 2 is depicted in FIG. 2, which shows the interior of housing 4. As shown, rubber gloves 14 are preferably attached to armrests 22 provided in the sidewall of housing 4. Armrests 22 are conveniently made from a steel or PVC tubing, e.g., a tube 6″ in diameter, 2.2″ in length, and having a typical wall thickness of ⅛″ if steel or ¼″ if PVC. The armrests are welded completely to housing 4 to fix them in place. The rubber gloves can be secured to the interior region of armrests 22 with a large hose clamp, and the like. The rubber gloves can be obtained from Comasec. Two UVC light bulbs 12, emanating light at 254 nm and 185, are located with one being positioned near the top of the chamber and the other being positioned under the grate 17. These UVC bulbs are available from GE. Grate 17 is also provided interior cylindrical housing 11 for supporting mail and/or packages thereon. Preferably, grate 17 rests on mounts 16 so that it slides out of cylindrical housing 11 upon the removal of endplate 6, allowing for easier servicing of interior palts. The grate is preferably about 47 inches long and about 17 inches wide. A visible light bulb 13 provides light for viewing. The visible light bulb is available from GE. A thin film coating 21 of TiO2 lines the entire inside walls of cylindrical housing 11. An electro-mechanical door interlock 20 engages when the sterilization cycle begins, thus locking door handle 8 and preventing the door 8 from opening until the sterilization cycle is completed and the UVC bulbs are de-energized. A normally open push-in switch, is mounted to the door interlock 20 in such a manner that the switch is depressed when the door closes, thus closing the switch. This door interlock is available from Edwards or RCI. A low volume fan 15, provides a slow but constant movement of air, ensuring sufficient contact time for complete sterilization and mineralization.

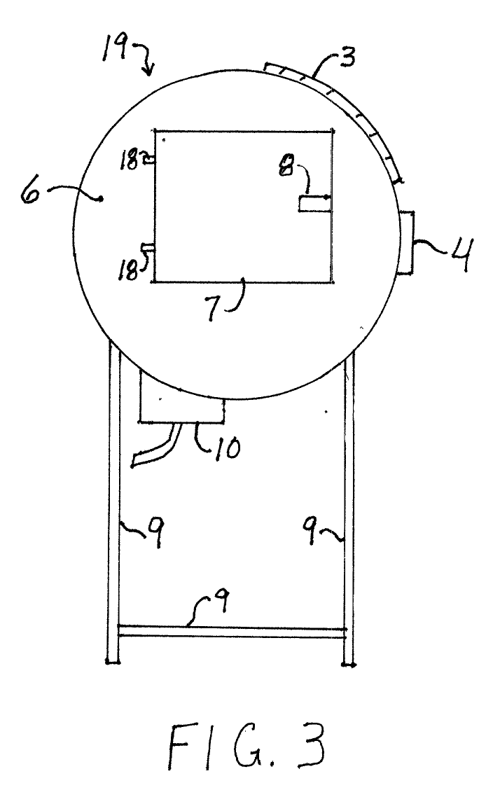

As shown in FIG. 3, a door 7 is provided at one end of cylindrical housing 11, through which mail and/or packages can be passed in or out of the housing. The door can be provided at either end of the housing, but as shown in FIGS. 1 and 3 is usually installed to the left of the user, who is usually right-handed. Left-handed models can be assembled as well. Thus, left end 19 is defined essentially by door 7, door 7 being attached to left end plate 6 by hinges 18, door handle 8 attached to door 7, and endplate 6, which partially covers end 19. Door 7 is gasketed beneath so that when compressed upon closing, an airtight seal of 1 atmosphere pressure is achieved. Endplate 6 is attached by bolting so that it can be removed for servicing interior parts. Endplate 6 is gasketed to an airtight seal of 1 atmosphere.

Opposing end 23 is depicted in FIG. 1 and is defined essentially by endplate 5, which is affixed to cylindrical housing 11, e.g., by welding. Welding can be with heat fusion if made of steel or cold fusion if made of plastic. Endplate 5 is typically devoid of any openings.

The present invention has been described above with reference to particular preferred embodiments for purposes of clarity and understanding. It should be appreciated that certain obvious additions and modifications to the present invention can be practiced within the scope of the appended claims. The illustrated embodiments are presented by way of example. The invention is not to be limited by such examples, but rather is defined by the claims.

We claim:

1. A device for sterilizing mail to ensure life-safety in the event of biological or chemical warfare agents hidden in mail and/or packages, comprising:
a horizontally arranged cylindrical housing having first and second ends and a curved sidewall, said housing having a longitudinal central axis extending horizontally through the interior of the housing, which curved sidewall is effective in reflecting photons toward said longitudinal axis;
a horizontally arranged cylindrical housing having first and second ends and a curved sidewall, said housing having a longitudinal central axis extending horizontally through the interior of the housing, which curved sidewall is effective in minimizing the distance from UVC bulbs to the target biologicals;
a horizontally arranged cylindrical housing having first and second ends and a curved sidewall, said housing having a longitudinal central axis extending horizontally through the interior of the housing, which curved sidewall being coated in a SiO2 thin film maximizes photocatalytic efficacy.
a first end-plate affixed to and partially covering a lower portion of the first end and a door that covers an upper portion of the first end not covered by the first end-plate;
a second end-plate affixed to and completely covering the second end;
means for supporting the cylindrical housing;
openings provided in the sidewall of the cylindrical housing through which a user's hands can be inserted;
an airtight covering into which the user's hands can be inserted (gloves) through the openings provided in the sidewall of the cylindrical housing;
a curved transparent window, which window blocks 100% of UV light, provided in the curved sidewall of the housing, which window is arranged relative to said openings so as to permit a user to view mail while manipulating the mail in the interior of the housing;
a UVC light source in the 254 nm wavelength;
an ozone generating light source in the 185 nm wavelength;
a chamber where the mail is positioned along the central longitudinal axis interior the housing;
a chamber wherein the interior surfaces of the cylindrical sidewalls are coated in a thin film of TiO2;
a chamber wherein the 254 nm UVC light enables a photocatalytic reaction;
a removable endplate to which a door is attached by hinges;
a handle attached to the door; and
an electro-mechanical door interlock with timer.

2. The device of claim 1, further comprising:
a grate provided interior the cylindrical housing for supporting mail and/or packages;
a light source in the visible spectrum which illuminates the contents of the cabinet;
electrical circuitry to supply current to the light sources, fan, and door interlock;
a normally open switch, attached to the door mechanism, opens when the door to the sealed cabinet opens, turning off the UVC light circuit;
a normally open switch closes when the door to the chamber closes, thus energizing the UVC light circuit; and
a timer circuit which is activated when the door closes, thus energizing the door locking mechanism, and disallowing the opening of the door until the timer cycle is completed, at which timer circuit de-energizes, thus unlocking the door.

3. The process for sterilizing mail and/or packages of biological agents and mineralizing chemical warfare agents defined in claims 1 through 2.
